(19) United States
(12) Patent Application Publication
(10) Pub. No.: US 2013/0108594 A1
(43) Pub. Date: May 2, 2013

(54) METHOD FOR EVALUATING ANGIOGENIC POTENTIAL

(71) Applicants: Enca Martin-Rendon, Headington, Oxford (GB); Suzanne Watt, Headington (GB)

(72) Inventors: Enca Martin-Rendon, Headington, Oxford (GB); Suzanne Watt, Headington (GB)

(73) Assignee: NHS BLOOD & TRANSPLANT, Watford, Hertfordshire (GB)

(21) Appl. No.: 13/643,966

(22) PCT Filed: Apr. 28, 2011
(86) PCT No.: PCT/GB2011/050856

§ 371 Date: Jan. 14, 2013

(30) Foreign Application Priority Data
Apr. 29, 2010 (GB) 1007159.5

(51) Int. Cl. G01N 33/68

(52) U.S. Cl. CPC G01N 33/6893
USPC 424/93.21; 435/7.1; 506/9; 435/6.12; 424/93.7; 435/377

(57) ABSTRACT

The invention provides a method for determining the angiogenic signature of a cell or tissue sample and methods for predicting efficacy of angiogenic therapy, for preparing a predictive model for predicting angiogenic potential, for identifying an agent for use in angiotherapy and therapeutic agents and applications.

The present invention relates to any ischaemic condition or disorders related to ischaemia and in particular coronary artery disease (CAD), peripheral arterial disease (PAD), stroke or cerebral ischaemia, wound healing or renal, lung or liver ischaemia. Specifically the invention relates to methods for evaluating the angiogenic potential of a subject or cells isolated from a subject, and adopting appropriate treatment regimens based on the evaluation, and to therapeutics.

Ischaemic heart disease (IHD) is a disease characterised by reduced blood supply to the heart muscle, usually due to coronary artery disease (CAD). In IHD the arteries that supply blood to the heart muscle become narrowed by atherosclerosis, a process where deposits of fatty, fibrotic, or calcified material build up on the inside of the artery. Atherosclerosis reduces the blood flow to the muscle of the heart which starves the muscle of oxygen, leading to angina pectoris, myocardial infarction, and congestive heart failure. Unlike many other tissues in the human body, heart tissue has a limited capacity for self-repair and ischaemic damage to the heart muscle can lead to an irreversible loss of myocardial tissue that leads to impairment of left ventricular function and ultimately heart failure. Despite the availability of therapeutic and interventional angioplastic treatments such as percutaneous coronary intervention (PCI) and coronary artery bypass graft (CABG), these established therapeutic options improve short-term survival of patients following myocardial infarction (MI), but do not stop the progression of the disease or the development of heart failure at later stages. The number of patients suffering from heart failure continues to increase and this remains a major cause of mortality and morbidity worldwide. For this reason, clinical research tends to be focussed on controlling symptoms and limiting or stopping the progression of the disease by replacing or regenerating the damaged tissue and improving revascularisation.

Peripheral arterial disease (PAD) is also characterised by reduced blood supply due to atherosclerosis, inflammation, embolism or thrombus formation that affects mainly the lower limbs. The affected arteries show sings of fat deposits or fibrotic or calcified material accumulation that narrow the arteries. It is caused by smoking, diabetes, dyslipidemia, hypertension and other cardiovascular risk factors. As in CAD, revascularisation is required to improve the patient's quality of life. However, in severe cases of PAD, revascularisation may not be sufficient and limb amputation is the only solution.

Revascularisation in patients with delayed wound healing due to large surface burns or chronic wounds affected by either arthrosclerosis or diabetes is also a major challenge.

Revascularisation or neural tissue damaged by stroke or focal or global cerebral ischaemia. Cerebral ischaemia results as insufficient blood flow to the brain occurs to meet metabolic demand. Ischaemia leads to alterations in brain metabolism, reduction of metabolic rates and energy crisis, with the subsequent damage to the brain tissue and possibly impairment in vision, speech and movements.

Angiogenesis is the natural healing process by which new blood vessels are formed to supply blood to an organ or damaged tissue. The process involves the budding of capillaries leading to the formation of new microvessels from pre-existing vascular structures. In the heart, angiogenesis can lead to the production of new blood vessels, known as collaterals, which can grow to bypass arterial blockages thereby providing an alternative source of blood supply to myocardial tissue jeopardised by ischaemia. Angiogenesis can be triggered by one or more mechanical, chemical, or molecular factors. Endothelial cells respond to physical forces and can convert mechanical signals into the molecular signals of angiogenesis. Augmentation of blood flow, for example during exercise, and increased shear stress and stretch on the myocardium may stimulate angiogenesis via increased production of growth factors.

Hypoxia (low oxygen tension) or ischaemia is a natural stimulus for angiogenesis. During hypoxia, the transcription factor hypoxia-inducible factor (HIF) binds to the hypoxia response elements (HREs) in the promoter region of hypoxia-responsive genes such as the vascular endothelial growth factor (VEGF) gene, enhancing its expression and release from the cell. Circulating VEGF then binds to VEGF receptors on the surface of endothelial cells, triggering a signal transduction pathway leading to angiogenesis. Hypoxia can also stimulate macrophages to release various factors including platelet-derived growth factor and fibroblast growth factor (FGF) 1 and 2, which also promote angiogenesis. Like VEGF, FGFs stimulate endothelial cell synthesis of proteases, including plasminogen activator and metalloproteinases, which are important for extracellular matrix digestion in the process of angiogenesis.

Angiogenesis may also be triggered through inflammatory processes. Following ischaemic damage to the myocardium, the influx of macrophages, monocytes, and platelets can lead to the release of cytokines and receptors that are capable of stimulating VEGF and FGF expression.

Although the mechanical, chemical and molecular factors associated with ischaemia up-regulate expression of angiogenic growth factors, this natural compensatory mechanism does not generate sufficient new blood vessels in every patient, notably in those patients exhibiting symptoms of IHD such as angina. Some patients with coronary artery disease develop poor collateral circulation, and others develop excellent collateral circulation. In those patients with poor collateral development, it has been suggested that the production of angiogenic cytokines may be inadequate, or the response to them may be attenuated (Freedman, S. B., and Isner, J. M; Annals of Internal Medicine 2002; 136(1): 54-71). Several factors are known to influence the expression of angiogenic cytokines and the angiogenic response, these accounting in part for the varied response to development of collateral vessels seen in patients. For example, animal studies demonstrate that older age and the existence of other medical conditions such as diabetes and hypercholesterolemia can lead to reduced VEGF expression and a lower angiogenic response (Rivard A., et al. Circulation. 1999; 99:111-20; Rivard A., et al. Am J. Pathol. 1999; 154:355-63; Couffinhal T, 2002 Circulation. 1999; 99: 3188-98). These factors are often relevant in patients with advanced coronary artery disease, who are often older and have diabetes, hypercholesterolemia, or other undetermined characteristics that limit up-regulation of angiogenic cytokines by ischaemia (Freedman, S. B., and Isner, supra). Beyond age and other disease conditions, VEGF regulation has been shown to vary between individuals, with VEGF expression in response to hypoxia being higher in monocytes from patients with good collateral development compared to patients with poor collateral development (Schultz A., et al., Circulation. 1999; 100:547-52). The extent of development of a collateral circulation during IHD appears to vary from individual to individual, with a number of medical and genetic factors coming into play. Angiogenesis can be exploited therapeutically to enhance or promote the development of collateral blood vessels in ischaemic tissue and as an alternative to high risk interventional treatments such as angioplasty, in particular PCI and CABG, or in combination with surgery to provide more complete revascularisation. Angiogenesis can also be exploited to improve the revascularisation of ischaemic tissue in patients suffering from stroke or cerebral ischaemia, in PAD patients, to avoid limb amputation in the more severe cases and in patients with delayed wound healing due to burns, arthrosclerosis or diabetes (for review, see Watt et al 2011; Critser and Yoder 2010; Martin-Rendon et al., 2009).

The adult postnatal vascular system is a complex one. Generally, angiogenesis is maintained in postnatal life during physiological and pathological conditions. The formation of new vessels is achieved by (i) angiogenesis or sprouting of endothelial cells (ECs) from pre-existing vessels, generally in response to ischemia, (ii) vasculogenesis or de novo differentiation of mature EC from endothelial progenitors cells residing in the bone marrow and (iii) arteriogenesis or increase of size of the lumen in pre-existing arterioles (Fischer et al., 2206).

Therapeutic angiogenesis can promote the development of collateral blood vessels in ischaemic tissues, (e.g. in coronary arterial disease (CAD), peripheral arterial disease (PAD), cerebral ischaemia) and delayed wound healing (for review, see Watt et al 2011; Critser and Yoder 2010; Martin-Rendon et al., 2009). Circulating endothelial precursors and Endothelial Colony Forming Cells (ECFC)/late outgrowth endothelial cells, are generally rare in normal adult human peripheral blood. They are not as easily detected as other cells in the blood, such as pro-angiogenic cells, and non-angiogenic cells (Estes et al., 2010).

Angiogenesis can be enhanced using protein, gene, or cell therapy. In protein therapy growth factors such as VEGF and bFGF are administered to the patient, and in gene therapy these can be sustainably expressed or inhibited in a targeted manner. Cell therapy involves the administration of e.g. embryonic stem (ES) cells, induced pluripotent stem (iPS) cells, cardiac, endothelial-, umbilical cord blood- or peripheral blood- or bone marrow-derived stem cells and progenitor cells to repair damaged heart tissue and promote angiogenesis. Stem cells are characterised by their ability to self-renew and differentiate into a diverse range of specialised cell types, including those required for repair of damaged heart tissue and formation of new blood vessels. Like stem cells, progenitor cells also have a capacity to differentiate into specific types of cells, however, progenitor cells are more differentiated and already committed to differentiation into certain cell types. Stem and progenitor cells from bone marrow and blood have been used in clinical trials to treat acute and chronic phases of myocardial infarction. They do exert a moderate but beneficial positive effect on the recovery of left ventricular function. However, the exact mechanism of action of stem and progenitor cell therapies is not fully understood, although it is thought that the cells may have beneficial effects on cardiac function by increasing vascularity via endothelial progenitor cell (EPC) incorporation into the ischaemic tissue, by generating cardiomyocytes, by modulating cardiac remodelling and/or in paracrine fashion by producing cytokines and or other factors that may promote cardiac repair and limit fibrosis in the affected area (Martin-Rendon E., et al., Transfusion Medicine 2009; 19:159-171). Consistent with the variation between individuals seen in the natural development of a collateral circulation, the response between individuals observed during angiogenic treatment also varies for genetic and epigenetic reasons (such as single nucleotide polymorphisms, microRNA expression and patterns of DNA methylation).

The angiogenic response arising due to ischaemia and/or therapeutic angiogenesis can vary between individuals due to a number of factors. Because of this known variability it is desirable to be able to predict the extent of natural development of collateral circulation in a patient with any ischaemic condition or disorders related to ischaemia and in particular coronary artery disease (CAD), peripheral arterial disease (PAD), stroke or cerebral ischaemia, wound healing or renal, lung or liver ischaemia and the extent of any response to therapeutic angiogenesis. Based on this prediction, appropriate treatment regimens can be designed on a patient by patient basis.

WO 2004/053085 discloses methods for identifying genes involved in angiogenesis by studying gene expression patterns in a mouse model with experimentally induced ischaemia (produced via femoral artery ligation). The disclosed method comprises isolating total RNA from adductor muscles, preparing cDNA and hybridising the cDNA to a gene array. Based on a comparison with gene expression levels associated with cDNA prepared from non-ischaemic mice, genes involved in angiogenesis were identified. This publication focuses on angiogenic gene expression in mice in an artificial model of ischaemia.

WO 2008/118846 discloses methods for predicting the angiogenic potential of a tumour in vivo. The method comprises culturing malignant cells from a patient specimen, and testing the cell culture for the presence and/or levels of: VEGF/VPF, bFGF/FGF-2, 1 L-8/CXCL8, EGF, Flt-3 ligand, PDGF-AA, PDGF-AA/BB, IP-10/CXCL10, TGF-β1, TGF-β2, TGF-β3, VEGFR, HIF1-alpha, EGFR, HER-2, TGF-alpha, TNF-alpha, thrombospondin, and angiogenin. The angiogenic signature of the tumour is then matched with anti-angiogenic treatment regimens and clinical outcomes for the patients from whom the specimens originated. This publication relates to tumour angiogenesis.

A report by Smith et al. (Smith et al., 2007) discloses methods for isolating and culturing cardiosphere-derived cells (CDC) from left ventricular biopsies from patients suffering from heart failure and who have received a heart transplant. The disclosed method comprises treating heart biopsies with mild enzymatic treatment, plating the explants in fibronectin-coated plates, collecting the outgrowth cells from the explants, culturing cells under specific conditions to form cardiospheres and expanding the cardiosphere-derived cells further on fibronectin-coated plates. The method also comprises characterising CDCs by flow cytometry and other standard immunofluorescence techniques and demonstrating their capacity to improve heart function by implanting CDCs in a rodent model of acute myocardial infarction. Based on these tests the therapeutic potential of human CDCs was assessed and the mechanism of how these cells may improve heart function suggested. The publication focuses on the isolation of CDCs, with a high proportion of c-kit⁺ cells, from the left ventricle in a particular cohort of patients and aims to demonstrate that these c-kit⁺ progenitor cells could repair the heart by differentiating into endothelial cells, vascular cells and cardiomyocytes.

The present invention provides a method for predicting the angiogenic potential of a subject suffering from any ischaemic condition or problems related to ischaemia and in particular suffering from coronary artery disease (CAD), peripheral arterial disease (PAD), stroke or cerebral ischaemia, delayed wound healing and renal, lung or liver ischaemia. The invention also provides a method for expanding endothelial cells and hence provide a method of treatment for any ischaemic conditions.

According to a first aspect of the present invention there is provided a method for determining the angiogenic signature of a cell or tissue sample comprising: with an ex vivo cell or tissue sample obtained from a subject (a) testing the sample for the expression levels of angiogenesis-related factors.

The factors may be pro-angiogenic proteins, anti-angiogenic proteins or proteins involved in cell-cell interaction or extracellular matrix deposition.

The angiogenesis-related factors may be selected from the group consisting of: ADAMTS-1 (A disintegrin and metalloproteinase with thrombospondin motifs 1), Angiogenin, Angiopoietin-1, Angiostatin, BMP-1 (Bone morphogenetic protein 1), EGF (Epidermal growth factor), EG-VEGF (Endocrine-Gland-Derived Vascular Endothelial Growth Factor), Endostatin, FGFa (Human Fibroblast Growth Factor-acidic), FGFb (Human Fibroblast Growth Factor-basic), PDGF-AA (Platelet-derived growth factor-AA), PDGF-AB (Platelet-derived growth factor-BB), Prolactin, TIMP-1 (Tissue inhibitor of metalloproteinase 1), pro-collagen type I and uPA (urinary Plasminogen Activator).

The method may comprise testing for the expression levels of at least one, at least two or at least three angiogenesis-related factors. The angiogenic signature may be a quantitative or semi-quantitative measurement.

The method may additionally comprise testing for expression levels of other factors related to apoptosis, cell cycle processes, cell surface processes, cell-cell interaction, cell migration, centrosomal processes, cellular adhesion, cellular proliferation, cytoskeletal processes, growth factors and receptors, membrane/integrin/signal transduction, proliferation, surface antigens and transcription factor molecular markers.

The expression levels of the angiogenesis-related factors may be compared to one or more control or reference levels of expression, which may be from the same or different subject, and before, during or after any treatment. The levels may be compared to levels determined for one or more control samples (e.g. cell cultures), or for one or more control factors that are not related to angiogenesis, these being well known in the art. The method may further comprise (b) comparing the results determined in step (a) with reference levels of expression; and (c) predicting the pro-angiogenic potential of the subject from the outcome of the comparison in step (b). The reference levels of expression may be derived from testing the expression levels of the factors in an ex vivo sample obtained from a different tissue from the same subject or the same or different tissue from another subject. The tissue may be adipose, cardiac, vascular or haematopoietic tissue. For example, a reference level may be the level(s) of the particular angiogenesis-related factors secreted from cultured cells or tissue derived from a patient known to be responsive, or not responsive, to a particular angiogenic therapy, or derived from a patient having a particular disease progression or angiogenic phenotype, for example one associated with a low pro-angiogenic potential, or a subject with suffering from any ischaemic condition or disorders related to ischaemia and in particular coronary artery disease (CAD), peripheral arterial disease (PAD), stroke or cerebral ischaemia, wound healing or renal, lung or liver ischaemia with no or insufficient natural development of collateral circulation. The reference levels of expression may be average or defined levels of expression associated with pro-angiogenic potential in one or more different subjects, where the reference levels of expression have been correlated with angiogenesis in an in vitro, in vivo or other functional angiogenesis assay. In its simplest embodiment, the method enables prediction of a subject's pro-angiogenic potential by determining the expression levels of certain angiogenesis-related factors and comparing the expression levels to reference levels which have been functionally linked to angiogenic potential. Differences in the levels of these factors are generally significant where the differences are at least about 1.5 fold, but may be 2, 5, 10, 20, 50 fold or more. Additional controls include testing the level of one or more factors that are not related to angiogenesis.

In a preferred embodiment the method may be used to predict the pro-angiogenic potential of a human subject suffering from ischaemic condition or disorders related to ischaemia and in particular coronary artery disease (CAD), peripheral arterial disease (PAD), stroke or cerebral ischaemia, wound healing or renal, lung or liver ischaemia. The expression levels may be correlated to disease state, aetiology, disease progression or risk factors such as diabetes, hypertension, hyperlipidemia, age, sex, obesity, body mass index, smoking, heart function and drug use.

Pro-angiogenic potential is defined as the tendency to stimulate blood vessel growth in tissues that require an improved blood supply. In subjects suffering from ischaemia, the method may predict the likelihood that a subject will develop collateral circulation or collaterals.

The applicant has experimentally determined that increased levels of one or more of ADAMTS-1, Angiogenin, Angiopoitin-1, Angiostatin, EGF, EG-VEGF, Endostatin, PDGF-AA, PDGF-AB, Prolactin, TIMP-1, pro-collagen type I and uPA compared to reference levels negatively correlate with pro-angiogenic potential. These factors are considered to be anti-angiogenic or associated with a low pro-angiogenic potential. The applicant has further determined that there is an inverse correlation between the ability of cardiac-derived cells to support angiogenesis and the expression of some extracellular matrix components (e.g. pro-collagen type I) in those cells. Furthermore, a correlation between cell proliferation and pro-angiogenic ability has been observed.

The applicant has experimentally determined that increased levels of one or more of FGFa, FGFb, and bone morphogenetic protein (BMP)-1 compared to reference levels positively correlate with pro-angiogenic potential. These factors are considered to be pro-angiogenic or associated with a high pro-angiogenic potential.

The angiogenic signature may be used to evaluate the angiogenic potential of the cells or tissue and to select an appropriate pro- or anti-angiogenic therapy.

In a second aspect the invention provides a method for predicting efficacy of angiogenic therapy, comprising: determining the pro-angiogenic potential of an ex vivo cell or tissue sample obtained from a subject using the method of the first aspect of the invention and matching the pro-angiogenic potential with a model profile correlated to angiogenic treatment regimen and subject outcome. The predictive method thus enables an appropriate treatment strategy to be devised based on the angiogenic potential of the cells or tissue obtained from the subject.

For subjects having predicted good or high pro-angiogenic potential, further therapeutic intervention than the standard ones (e.g. drugs, PCI or CABG) may not be required. For those subjects predicted to have low or poor pro-angiogenic potential, therapeutic angiogenesis or other treatment regimens may be indicated as complementary to standard treatments.

In certain embodiments, the method of the invention tests for several pro-angiogenic angiogenesis-related factors (e.g. two, three, or four), and optionally, one or more (e.g. two) anti-angiogenic angiogenesis-related factors.

In some embodiments, computer algorithms may be used to compare the pro-angiogenic potential with a model profile.

In a preferred embodiment the method may be used to predict the clinical response of a human subject suffering from any ischaemic condition or disorders related to ischaemia and in particular coronary artery disease (CAD), peripheral arterial disease (PAD), stroke or cerebral ischaemia, wound healing or renal, lung or liver ischaemia towards angiogenic therapy. The model profile may be correlated to disease state, aetiology, disease progression or risk factors such as diabetes, hypertension, hyperlipidemia, age, sex, obesity, body mass index, smoking, heart function and drug use. The method may provide a probability of response to therapeutic angiogenesis or future risk of disease progression for the subject.

In a third aspect the invention provides a method for preparing a predictive model for predicting angiogenic potential, comprising: with ex vivo cell or tissue samples obtained from a plurality of subjects, (a) testing each sample for the expression levels of at least three angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA; (b) testing the each sample in a functional assay; and (c) matching the results from step (a) with the results from step (b).

By functionally assessing the angiogenic ability of each sample with the expression levels of one or more angiogenesis-related factors, reference expression levels of each factor can be determined, and correlated with angiogenesis. The assay may be an in vitro and/or in vivo angiogenesis assay. In vivo angiogenesis assays may be based on the use of Matrigel implants in immunocompromised mice as described by Passaniti A., et al. (Lab Invest. 1992 October; 67(4):519-28). An extract of basement membrane proteins (Matrigel) can be reconstituted into a gel when implanted subcutaneously into mice. Matrigel can be seeded with e.g. endothelial cells and supportive cells prior to implantation. New vessels are formed in vivo which link up with the mouse blood vessels. Angiogenic factors or inhibitors can also be added to the Matrigel. The Matrigel plug can then be removed for immunohistochemical analysis, allowing assessment of the number of vessels or vessel density.

In vitro, the assay may test the ability to form cardiospheres, vascular endothelium, and pro-angiogenic supportive cells. The assay may comprise the use of human endothelial cells (e.g. human umbilical vein endothelial cells (HUVEC) or human microvascular endothelial cell (HMEC)) or endothelial progenitor cells (EPC) or endothelial colony forming cells (ECFC) obtained from ES cells, iPS cells, haematopoietic tissues such as umbilical cord or peripheral blood or adult vessels or microvasculature which may be co-cultured with mesenchymal stem cells, stromal cells or supportive cells obtained from umbilical cord, umbilical cord blood, Wharton's Jelly, peripheral blood, bone marrow, skin, adipose tissue or cardiac or skeletal muscle derived cells in endothelial media that allows the formation of vascular tubular structures. Endothelial, blood or bone marrow cells (e.g. from consented donors) may be used as controls for vascular formation.

In a fourth aspect, the invention provides a method for identifying an agent for use in angiotherapy, comprising: with an ex vivo cell or tissue sample obtained from a subject, (a) contacting the sample with the agent, (b) testing the sample for the presence and/or levels of at least three angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA; (c) comparing the presence and/or levels of expression of the angiogenesis-related factors with the presence and/or levels of expression of the same angiogenesis-related factors in a sample that has not been contacted with the agent. A statistically significant change in expression of one or more angiogenesis-related factors in the presence of the agent relative to an expression level in the absence of the candidate agent may be indicative of activity of the agent in modulating angiogenesis. An increase in expression of one or more pro-angiogenic angiogenesis-related factors in the presence of the agent relative to expression levels in the absence of the agent may indicate that the agent has pro-angiogenic activity and a decrease in expression of one or more pro-angiogenic angiogenesis-related factors in the presence of the agent relative to expression levels in the absence of the agent may indicate that the agent has anti-angiogenic activity. The method may comprise obtaining an angiogenesis-related factor expression profile from a cell or tissue sample contacted with an agent; comparing the obtained expression profile to a reference angiogenesis-related factor expression profile to determine whether the candidate agent has activity in promoting or inhibiting angiogenesis. The expression profile may be a transcriptional profile or a protein profile.

The invention also provides kits for carrying out any of the methods described above.

The invention further provides a kit for screening an agent for activity in modulation of angiogenesis, the kit comprising: a set of nucleic acid primers, polynucleotide hybridisation probes, or one or more antibodies specific for one or more angiogenesis-related factors. The reagents may be located on a surface in an array. The kit may further comprise reference levels of expression or one of more model profiles, and instructions for use.

The cell or tissue sample may be obtained via a biopsy specimen from a healthy subject or a subject in need of treatment, suffering from any ischaemic condition or disorders related to ischaemia and in particular coronary artery disease (CAD), peripheral arterial disease (PAD), stroke or cerebral ischaemia, wound healing or renal, lung or liver ischaemia. The sample may be obtained from the subject's heart or skeletal muscle, blood vessels, microvasculature or associated vascular tissue, adipose tissue, umbilical cord, umbilical cord blood, peripheral blood or bone marrow or from the subject's embryonic cells or iPS cells or cell lines generated by standard methods known in the art. The sample may be an atrial or ventricular biopsy, a skeletal muscle biopsy or a biopsy from a venous or arterial blood vessel or microvessel or adipose tissue, which may be obtained during revascularisation procedures, cardiac surgery or PCI, during skin replacement, wound healing or adipose tissue removal. The sample may be obtained from venous or arterial peripheral blood. The sample may comprise cardiac, skeletal, adipose tissue, endothelial or stromal supportive cells or mesenchymal or haematopoietic stem or progenitor cells. The samples may comprise iPS cells derived from any of the above by standard methods know in the art.

The cardiac cells may be cardiac-derived progenitor cells or stem cells, cardiomyocytes or associated cardiac stromal cells, fibroblasts or vascular cells or iPS cell-derived cardiac cells. The bone marrow-derived mesenchymal stem cells or haematopoietic stem cells may be used as control cells, to provide reference levels of expression. In all aspects, cells may be isolated and expanded from any sample using conventional techniques known in the art. More than one sample may be obtained from a subject, from different tissues for comparison purposes.

The expression level of angiogenesis-related factors may be determined in cell culture media or cell extracts. Preferably the testing provides quantitative or semi-quantitative results, which facilitate comparison with reference levels of expression or a model profile correlated to angiogenic treatment regimen and subject outcome. The testing may comprise measuring the presence and/or expression levels of the factor, either within the cell or secreted into the cell culture medium, using any suitable assay, such as an antibody-based assay, for example Western blotting or immunocytochemistry, but preferably using quantitative immunoassays such as ELISA. Kits for measuring levels of many proteins using ELISA methods are commercially available (e.g. from R&D Systems) and ELISA methods can be developed using well known techniques, e.g. as disclosed in “Antibodies: A Laboratory Manual” (Harlow and Lane Eds. Cold Spring Harbor Press). Antibodies for use in such ELISA methods either are commercially available or may be prepared using well known methods.

The testing may comprise measurement of the levels of gene expression at the mRNA level, using quantitative mRNA amplification methods such as RT-PCR, isothermal nucleic acid amplification, or variants thereof. Systems for carrying out these methods also are commercially available, for example the TaqMan system (Roche Molecular System, Alameda, Calif.) and the Light Cycler system (Roche Diagnostics, Indianapolis, Ind.). Methods for devising appropriate primers for use in RT-PCR and related methods are well known in the art. Angiogenesis-related factor or other factor protein expression levels may be correlated with mRNA levels by quantitative RT-PCR.

Nucleic acid arrays may be used to study the expression of one or more angiogenesis-related factors. In particular, arrays provide a method for simultaneously assaying expression of a large number of genes. Such methods are now well known in the art and commercial systems are available from, for example, Affymetrix (Santa Clara, Calif.), Incyte (Palo Alto, Calif.), Research Genetics (Huntsville, Ala.) and Agilent (Palo Alto, Calif.). The invention further provides an array of polynucleotide probes, the array comprising a support with at least one surface and a plurality of different polynucleotide probes, wherein each different polynucleotide probe hybridizes under stringent hybridization conditions to a gene product of a set of angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA.

Other methods of quantitative analysis of protein expression levels include proteomics technologies such as isotope coded affinity tag reagents, MALDI TOF/TOF tandem mass spectrometry and 2D-gel/mass spectrometry technologies. In one preferred embodiment cell extracts may be used to probe a proteome array (e.g. Proteome Profiler Array obtained from R&D Systems) that contains capture antibodies specific to one or more angiogenesis-related factors. The invention further provides an array of antibodies specific for one or more angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA.

Prior to testing the cells may be isolated from tissue using conventional separation and differentiation techniques. Preferably the cells are expanded in cell culture. The methods of the invention may comprise culturing the cells from the sample obtained from the each subject. The cells from the cell culture may be exposed to agonists or antagonists of angiogenesis. The cells may be maintained under normoxic or hypoxic culture conditions. The cells may be maintained in hypoxic and normoxic culture conditions and exposed to agonists or antagonists of angiogenesis in sequence or in parallel, and the expression levels of the angiogenesis-related factors may be tested in both. A hypoxic condition or environment may be about 0.5% to about 15% oxygen, preferably from about 1% to about 5% oxygen. Normoxic conditions include conditions at about 18% to about 23% oxygen, preferably about 21%.

In preferred embodiments, the cells obtained from the subject are expanded in culture and tested for expression levels of one or more angiogenesis-related factors.

Where the expression of only a relatively small number of factors is studied, changes in expression in most or all of those factors may need to be observed to provide a reliable prediction of pro-angiogenic potential. For example, where expression levels of only three factors are tested, changes in expression of all three factors may be reliably predictive of pro-angiogenic potential. Where expression levels of five factors are tested, changes in expression of at least four factors may provide a reliable prediction of pro-angiogenic potential. It is preferred that the expression level of a greater number of angiogenesis-related factors is tested to overcome any heterogeneity of expression, thereby increasing the confidence with which predictions of pro-angiogenic potential can be made. The methods of the invention may comprise testing for the expression levels of at least four, at least five, at least six, at least seven, at least eight, at least nine, at least ten, at least eleven, at least twelve, at least thirteen, at least fourteen, at least fifteen, or all of ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA are tested.

The subject is preferably human but may be a non-human mammal. The subject may be healthy, or may be suffering from any ischaemic condition or disorders related to ischaemia and in particular coronary artery disease (CAD), peripheral arterial disease (PAD), stroke or cerebral ischaemia, wound healing or renal, lung or liver ischaemia.

For those subjects determined or predicted to have low or poor pro-angiogenic potential, it is desirable to improve the pro-angiogenic potential of the cells and their expanded progeny, which are preferably cardiac-, or skeletal muscle-, vascular-, adipose tissue-, Wharton's Jelly, umbilical cord, umbilical cord blood, peripheral blood- or bone marrow-derived cells, stem cells, progenitor cells, ES cells or iPS cells. Strategies to improve the pro-angiogenic potential include hypoxia preconditioning, drug, small molecule or peptide treatment and/or genetic modification.

The invention also provides a method for improving the pro-angiogenic potential of cells, comprising treating an ex vivo population of cells obtained from a subject and/or expanded in culture using one or more of (i) hypoxia preconditioning, (ii) pharmacological treatment (iii) recombinant polypeptides and (iv) genetic modification.

There is provided a method for improving the pro-angiogenic potential of cells comprising: with an ex vivo cell or tissue sample obtained from a subject (a) testing the sample for the expression levels of at least three angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA; (b) comparing the results determined in step (a) with reference levels of expression; (c) predicting the pro-angiogenic potential of the subject from the outcome of the comparison in step (b), optionally expanding the cells in the sample in cell culture and (d) treating the cells using one or more of (i) hypoxia preconditioning, (ii) pharmacological treatment and (iii) recombinant polypeptides and (iv) genetic modification.

There is provided a cell or cells obtainable by the method.

The method may further comprise transplantation of the treated cells into the subject.

The invention further provides the use of a cell having improved pro-angiogenic potential in the treatment of any ischaemic condition or disorders related to ischaemia and in particular coronary artery disease (CAD), peripheral arterial disease (PAD), stroke or cerebral ischaemia, wound healing or renal, lung or liver ischaemia.

In a preferred embodiment, cardiac- or skeletal muscle-, vascular-, adipose tissue-, Wharton's Jelly, umbilical cord, umbilical cord blood, peripheral blood- or bone marrow-derived cells, stem cells, progenitor cells, ES cells or iPS cells may be cultured at about 1% to 5% oxygen (hypoxia) to enhance stem cell proliferation or differentiation potential. The cells may be contacted with a composition that decreases expression of one or more of: ADAMTS-1, Angiogenin, Angiopoitin-1, Angiostatin, EGF, EG-VEGF, Endostatin, PDGF-AA, PDGF-AB, Prolactin, TIMP-1, pro-collagen type I and uPA; and/or increases expression of one or more of: FGFa, FGFb, and BMP-1. The composition may contain an antisense oligonucleotide, an siRNA molecule, an RNAi molecule, an oligonucleotide that binds to mRNA to form a triplex, or a DNA molecule that is transcribed in the cells to produce an antisense oligonucleotide, an siRNA molecule, an RNAi, or an oligonucleotide that binds to mRNA to form a triplex, any of which may be obtained from a library, or via library screening. The composition may contain an antibody or a soluble protein receptor, for example, a human antibody or a human soluble protein receptor, which binds to an anti-angiogenic protein or a combination of those.

The cells may be contacted with one or more ACE inhibitors, ACE receptor antagonists, statins, or any other drug or active factor during culture to increase cell numbers, enhance survival and improve pro-angiogenic activity.

The present invention provides a method for determining the expression of certain angiogenesis-related factors to predict a subject's potential to develop collateral circulation or improve microcirculation naturally or in response to angiogenesis therapy.

In a further aspect the invention provides a method for expanding endothelial cells and ECFS in vitro and/or in vivo, comprising treating the EC or ECFC from an embryo, a neonate or adult tissue, such as umbilical cord, umbilical cord blood, peripheral blood or microvasculature, ES- or iPS-derived endothelial cells or ECFCs, with one or a combination of angiogenesis-related factors that enhance or augment cell numbers or support cell survival. Such factors could be one of ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA;, preferably BMP-1 or a combination of BMP-1 and other pro-angiogenic factors (e.g. ADAMTS-1, Angiogenin, Angiopoietin-1, Angiostatin, EGF, EG-VEGF, Endostatin, FGFa, FGFb, PDGF-AA, PDGF-AB, Prolactin, TIMP-1, pro-collagen type I and uPA;).

The method may include treating the endothelial cells with recombinant BMP-1 polypeptide or protein during culture ex vivo or delivery of recombinant BMP-1 protein or polypeptide as pro-angiogenic therapy or treatment for any ischaemic condition or disorders related to ischaemia and in particular coronary artery disease (CAD), peripheral arterial disease (PAD), stroke or cerebral ischaemia, wound healing or renal, lung or liver ischaemia, where increased collateral vessel growth is required. The delivery of the treatment may include encapsulating the recombinant BMP-1 protein for slow release, delivering BMP-1 in nanoparticles or implanting the capsules in live or artificial tissue grafts or coating stents.

The method may also include genetically modifying endothelial cells or ECFCs to express BMP-1 temporarily or stably using either plasmid DNA or viral vectors such as adenovirus-, adeno associate virus-, retrovirus- or lentivirus-based vectors under the control of a ubiquitous or inducible promoter for optimal gene expression.

The method may also include treating the endothelial cells or ECFCs with the supernatant or conditioned medium obtained from mesenchymal or stromal supportive cells from any of the tissues selected from embryo, a neonate or adult tissue, such as umbilical cord, umbilical cord blood, peripheral blood or microvasculature, ES- or iPS-derived endothelial cells or ECFCs. The supportive cells are preferably genetically modified to express BMP-1 temporarily or stably using one of the vectors mentioned above under the control of a ubiquitous or inducible promoter for optimal gene expression or treated with an agent that increases the expression of BMP-1 temporarily or stably.

The method may also include delivering the genetically modified or treated cells expressing BMP-1 or BMP-1 and a combination of pro-angiogenic factors, as treatment, directly to the damaged tissue to increase the number or the survival of endothelial cells in situ or the growth of collateral vessels.

The method may include improving collateral growth or improving microvasculature integrity in any ischaemic condition or disorders related to ischaemia and in particular coronary artery disease (CAD), peripheral arterial disease (PAD), stroke or cerebral ischaemia, wound healing or renal, lung or liver ischaemia.

As endothelial progenitor cells (EPCs) and ECFCs occur in low numbers postnatally, the challenges here would be to generate them in large quantities for therapeutic angiogenesis. Autologous transplantation of ex vivo expanded EPCs or ECFCs, incorporated into engineered grafts or stents, could be used to promote re-vascularisation of ischaemic tissues in any ischaemic condition or disorders related to ischaemia and in particular coronary artery disease (CAD), peripheral arterial disease (PAD), stroke or cerebral ischaemia, wound healing or renal, lung or liver ischaemia. EPCs and ECFC can be used as cellular therapies as factories for re-vascularisation to rescue limb ischaemia and promote healing, or to deliver one or more pro-angiogenic factors. These cells can also be used in combination with supportive cells for tissue engineering applications to create a microvasculature. The supportive cells, such as bone marrow or tissue specific mesenchymal progenitor or stromal cells can be used on their own as vehicles of one or more angiogenesis-related factors to induce the proliferation and/or survival of EC and ECFC and the formation of new blood vessels during angiogenic therapy. These supportive cells could be of autologous or allogeneic origin. Finally, ex vivo expanded EC and ECFC could be used for re-endothelialization of damaged vessels and maintenance of endothelial integrity on their own, in tissue grafts or stents.

In a further aspect of the invention there is provided the use of expanded EC and ECFCs for the preparation of a medicament for use in the treatment of any ischemic conditions or in angiogenic therapy. Ischemic conditions include for example, coronary artery disease (CAD), peripheral arterial disease (PAD), stroke or cerebral ischaemia, wound healing, and renal, lung or liver ischaemia.

Expanded cells may be used as factories for BMP-1 and other angiogenesis-related factors such as ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA. Expanded cells may be seeded into live or acellular or artificial tissue grafts or coating stents. Expanded cells may be used for re-endothelialisation.

Treated or genetically modified supportive cells such as mesenchymal or stromal cells on their own or in combination with EC and ECFCs may be seeded into live or acellular or artificial tissue grafts or coating stents

In a still further aspect of the invention there is provided the use of angiogenesis-related factors such as BMP-1 or BMP-1 and a combination of other pro-angiogenic factors for the preparation of a medicament for the treatment of any ischemic condition. The treatment may include the administration of the polypeptide or recombinant proteins directly into ischemic tissues. BMP-1 or BMP-1 and a combination of other angiogenesis-related factors could be delivered as part of live or acellular or artificial tissue grafts or coating stents. BMP-1 or BMP-1 and a combination of other angiogenesis-related factors could be delivered by a cell that is a supportive cell. Supportive cells include bone marrow, umbilical cord blood, Wharton's Jelly, perivascular, adipose tissue, cardiac tissue cells, microvascular-derived mesenchymal/stromal cells and adult and embryonic skin fibroblasts, embryoinic stem (ES) cells or induced pluripotent stem (iPS) derived supportive stromal cells.

According to a further aspect of the invention there is provided the use of expanded EC or ECFCs for the preparation of a medicament for the treatment of any ischaemic condition. Preferably the ischemic conditions may be selected from coronary artery disease (CAD), peripheral arterial disease (PAD), stroke, cerebral ischaemia, wound healing, liver, lung or renal ischaemia.

In a preferred embodiment, the EC or the ECFC may be modified so as to produce angiogenesis-related factors select from ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA.

Conveniently, the EC or ECFC may facilitate re-endothelialisation in a subject with an ischemic condition.

Routes of administration include topical, thin film, transdermal patch, intramuscular injection, intracoronary, intravenous or intra-arterial infusion.

In another aspect of the invention there is provided a pharmaceutical composition comprising a angiogenesis-related factor such as BMP-1 polypeptide and a pharmaceutically acceptable excipient, diluent or carrier. The angiogenesis-related factor may be encapsulated for slow or fast release. Any suitable components known in the prior art may be used for preparing the pharmaceutical composition.

BMP-1 or BMP-1 and a combination of other angiogenesis-related factors may be used to enhance survival of pro-angiogenic supportive cells such as mesenchymal or stromal cells.

The composition may comprise one or more of expanded EC, ECFC, BMP-1 or BMP-1 and one or more other pro-angiogenic factor and a pharmaceutically acceptable diluent, carrier or excipient.

Preferably the composition is in the form of a solid, liquid or in suspension and may be formulated for slow release over a short or long term.

In another aspect of the invention, BMP-1 or a combination of BMP-1 and one or more other angiogenesis-related factors may be used in medical treatments or therapy. Such treatments may include treatment of ischemic conditions.

Preferably, the ischemic condition is selected from coronary artery disease (CAD), peripheral arterial disease (PAD), stroke, cerebral ischaemia, wound healing, liver, lung or renal ischaemia.

The composition or treatment may be administered in any suitable form. Suitable administration forms may be prepared by mixing the active ingredient such as BMP-1 peptide with a conventional pharmaceutically acceptable carrier, excipient, binder, stabilizer, etc. When administered in the form of an injection, a pharmaceutically acceptable buffering agent, solubilizer, isotonic agent, etc. may be added thereto.

The dosage may vary according to the symptoms, ages, body weights, the administration form, the frequency of the administration, etc., but it may be in the range of 0.0001 to 3000 mg per day for an adult, which is administered once or divided into several dosage units. The dosage may be increased or decreased according to needs of the patient.

Compositions suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Liquid formulations include suspensions, solutions, syrups and elixirs. Liquid formulations may also be prepared by the reconstitution of a solid, for example, from a sachet.

Orally administrable compositions may be in the form of oral solid compositions, such as tablets, capsules, pastilles, pellets, pills, lozenges powders and granules. The composition may be in solid form which melts on contact with the tongue of the patient, for example in the form of disintegrating tablets. Shaped oral compositions are preferred, since they are more convenient for general use.

Solid forms for oral administration are usually presented in a unit dose, and contain conventional additives such as adjuvants, binding agents, diluents, disintegrants, dispersing agents, excipients, fillers, tabletting agents, lubricants, colorants, flavourings, desiccants, humectants, and wetting agents.

Pills, pellets and tablets may be coated according to well known methods in the art. Oral solid formulations also include conventional sustained release formulations, such as tablets or granules having an enteric coating.

Suitable fillers include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

Solid oral compositions are prepared by admixture, and may be prepared by conventional methods of blending, filling, tabletting or the like. Repeated blending operations may be used to distribute the active agent throughout those compositions employing large quantities of fillers. Such operations are, of course, conventional in the art.

Compositions suitable for parenteral administration include injectable and infusible aqueous or oily blends, mixtures, suspensions, solutions, emulsions and low-viscosity gel preparations. Compositions for parenteral administration may be formulated to be immediate and/or modified release. Modified release formulations include delayed-, sustained-, pulsed-, controlled-, targeted and programmed release.

The compositions may also contain adjuvants such as suspending agents, for example methyl cellulose, gelatin, hydroxyethylcellulose, carboxymethyl-cellulose, aluminium stearate gel, hydrogenated fats; emulsifying agents, for example lecithin, sorbitan monooleate, or acacia; preservatives, for example methyl or propyl phydroxybenzoate or sorbic acid; polysorbates, for example Tween 80 and if desired conventional colouring agents and wetting agents; and inert diluents such as sucrose, lactose or starch.

In another aspect of the invention there is provided a medical implant comprising one or more of expanded EC, ECFC or an angiogenesis-related factor such as ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA.

The EC or ECFC may be prepared by the method described in the invention.

The implant may be an artificial tissue grafts, stents live tissue grafts, acellular tissue grafts, a transdermal patch, or a thin film,

The implant preferably includes an angiogenesis-related factor selected from BMP-1 or a combination of BMP-1 and one or more other angiogenesis-related factor.

The invention will now be demonstrated by way of example only by the following figures and examples in which:

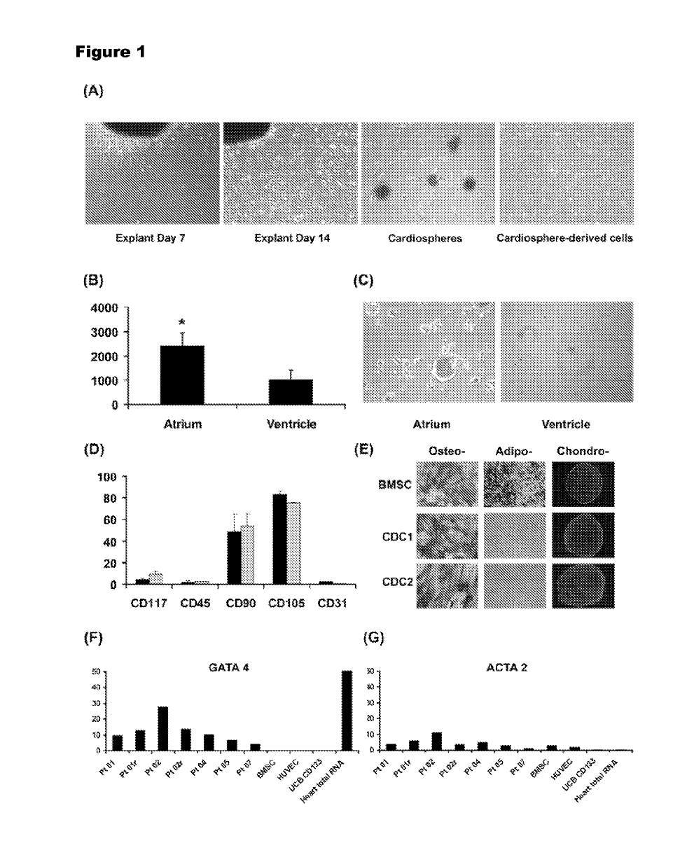

FIG. 1 shows Cardiac-derived cell characterisation. (A) Representative images of explants, cardiospheres and cardiosphere-derived cells (CDC) in culture on fibronectin-coated plates. (B) Number of phase-bright cells from atrial and ventricular biopsies of 28 patients suffering from ischaemic heart disease. (C) Representative images of cardiospheres from atrial and ventricular biopsies. (D) Expression of cell surface markers determined by flow cytometry. (E) Representative images of osteo-, adipo- and chondrogenic differentiation of CDCs from two patients (CDC1 and CDC2) compared to bone marrow mesenchymal progenitors (BMSC). (F) Expression of GATA 4 and (G) ACTA 2 (α-smooth muscle actin) assessed by RT-PCR in five patients (Pt), as examples of cardiac and smooth muscle cell markers. Expression is reported relative to β-2-microglobulin, a house-keeping gene (in percentage, %). Total mRNA from bone marrow mesenchymal stromal cells (BMSC), human umbilical vein endothelial cells (HUVEC), umbilical cord blood (UBC) CD133-positive cells and human heart were used as controls for RT-PCR analysis.

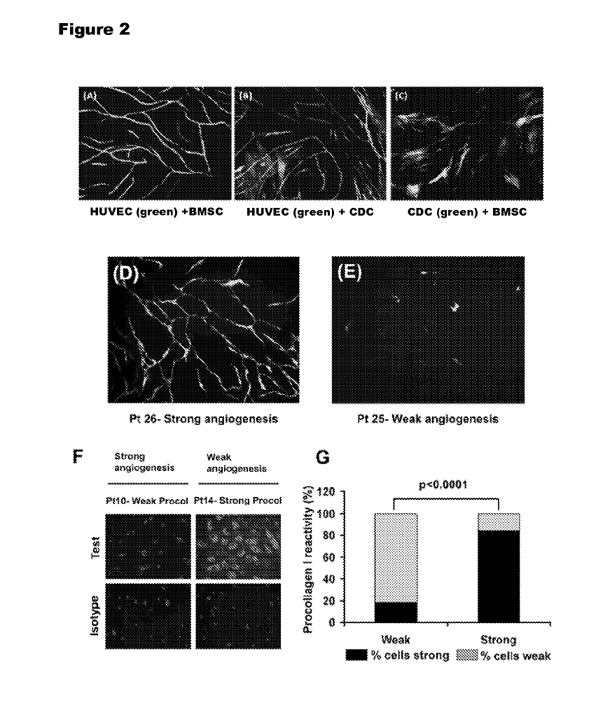

FIG. 2: Pro-angiogenic potential of cardiosphere-derived cells in vitro. (FIG. 2A) Representative image of GFP-labelled human umbilical vein endothelial cells (HUVEC) co-cultured with bone marrow mesenchymal stem cells (BMSC), (FIG. 2B) image of GFP-labelled HUVEC with cardiosphere-derived cells (CDC) and (FIG. 2C) GFP-labelled CDC with BMSC. Representative images of blood vessels supported by CDC with ‘strong’ (patient 26—Pt 26) (FIG. 2D) and ‘weak’ (patient 25—Pt 25) (FIG. 2E) pro-angiogenic potential are shown respectively. (FIG. 2F) Representative images of pro-collagen type I immunoreactivity (in red) and nuclei (in blue) compared with angiogenic potential of CDC (patients 10 and 14 as examples—Pt 10 and Pt14). (FIG. 2G) Pro-collagen type I immunoreactivity in CDC obtained from 10 patients is significantly different between the ‘weak’ and ‘strong’ groups.

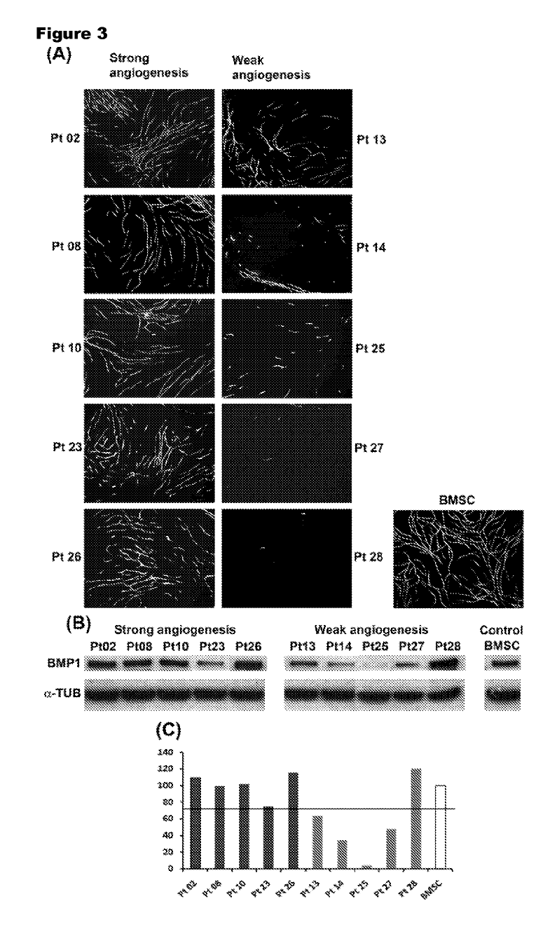

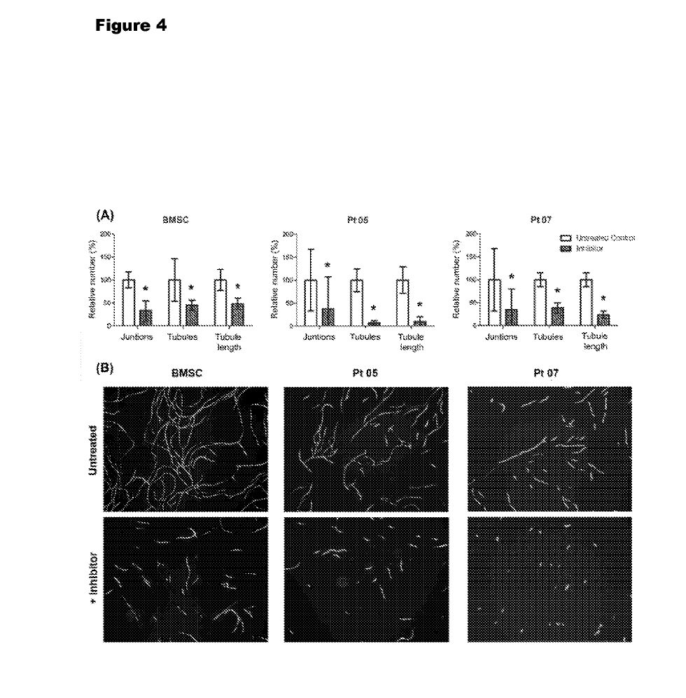

FIG. 3: Pro-angiogenic potential of cardiac-derived cells (CDCs) from patients with ischaemic heart disease. (A) Representative images of vascular structures in vitro. (B) Western blot showing the expression of bone morphogenetic protein (BMP) 1 protein in CDCs obtained from the patients above and in bone marrow-derived mesenchymal stromal cells (BMSC). Relative BMP-1 expression levels in CDCs compared with control cells BMSCs (C).

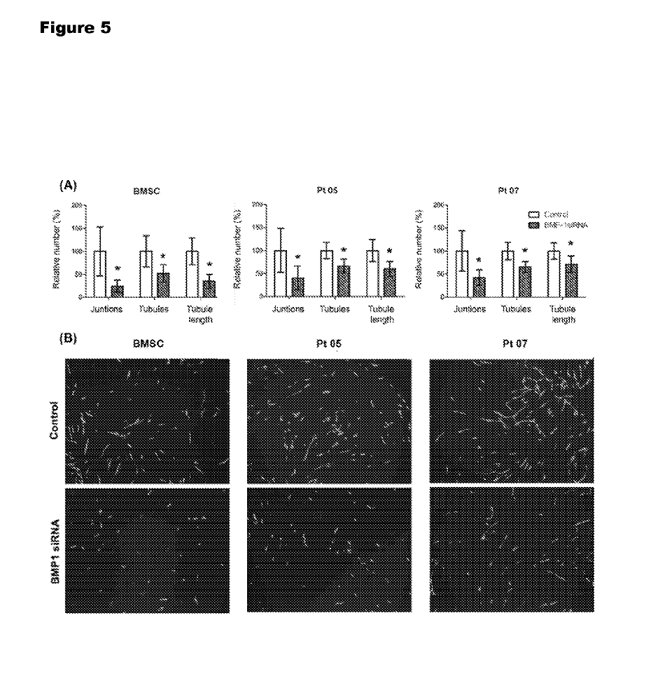

FIG. 4: Inhibiting BMP1 activity reduces blood vessel formation. (A) Relative angiogenesis in the absence (control-white bars) or presence (+inhibitor-dark bars) of BMP1 specific inhibitors (B) Representative images of the co-cultures on day 14 in the absence and presence of BMP1 inhibitor.

FIG. 5: BMP1 knock-down by siRNA reduces blood vessel formation. (A) Relative angiogenesis in cells treated with either non-target control (control-white bars) or specific BMP1 (dark bars) siRNA oligos. (B) Representative images of vascular network taken at day 4.

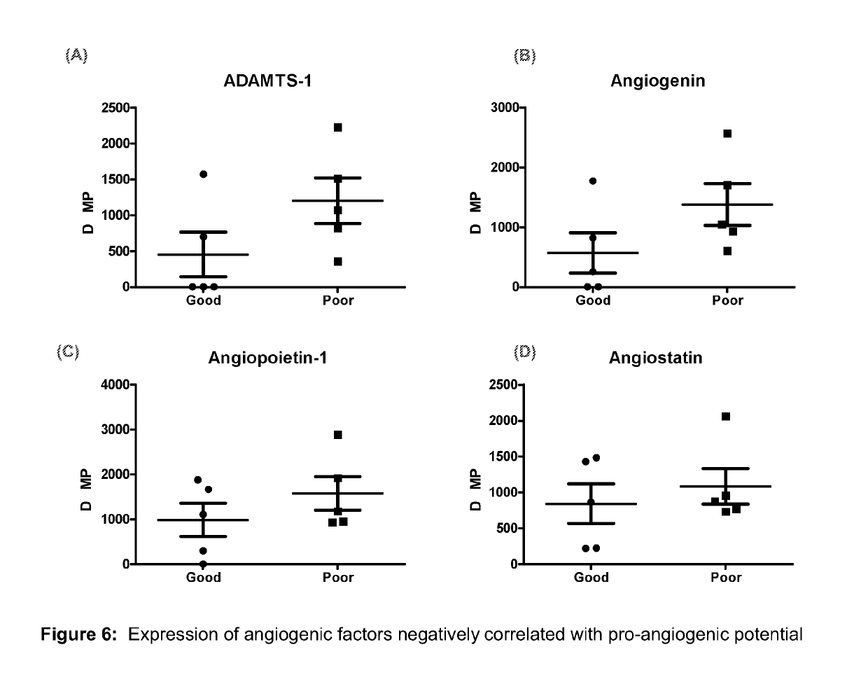

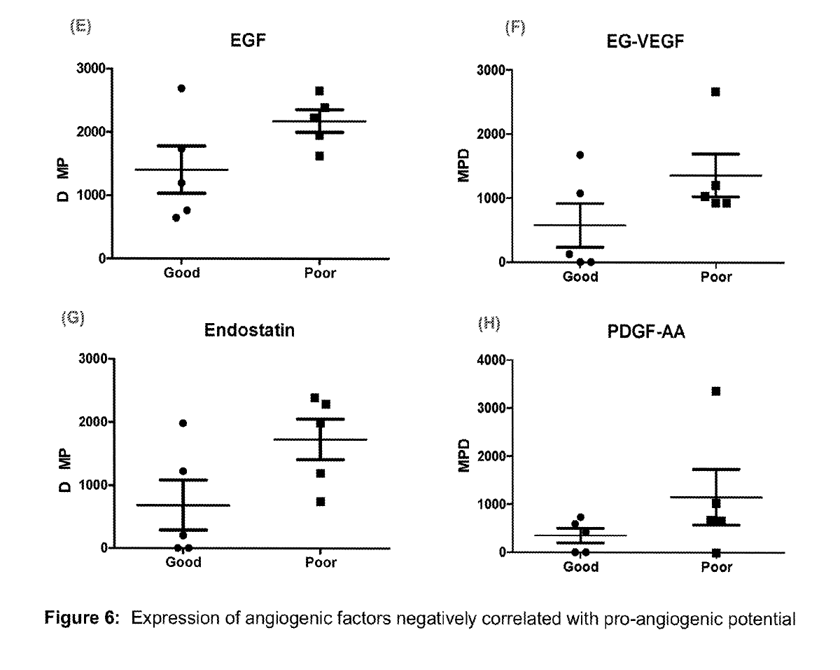

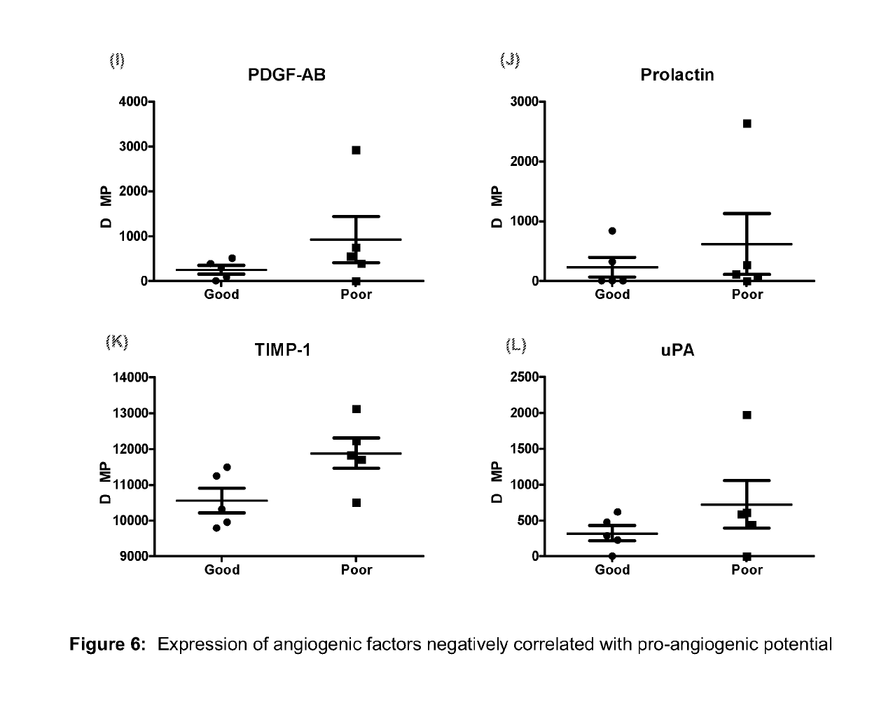

FIG. 6: Differential expression of angiogenic factors that negatively correlate with the cells pro-angiogenic potential: ADAMTS-1 (FIG. 6A), Angiogenin (FIG. 6B), Angiopoitin-1 (FIG. 6C), Angiostatin (FIG. 6D), EGF (FIG. 6E), EG-VEGF (FIG. 6F), Endostatin (FIG. 6G), PDGF-AA (FIG. 6H), PDGF-AB (FIG. 6I), Prolactin (FIG. 6J), TIMP-1 (FIG. 6K), and uPA (FIG. 6L).

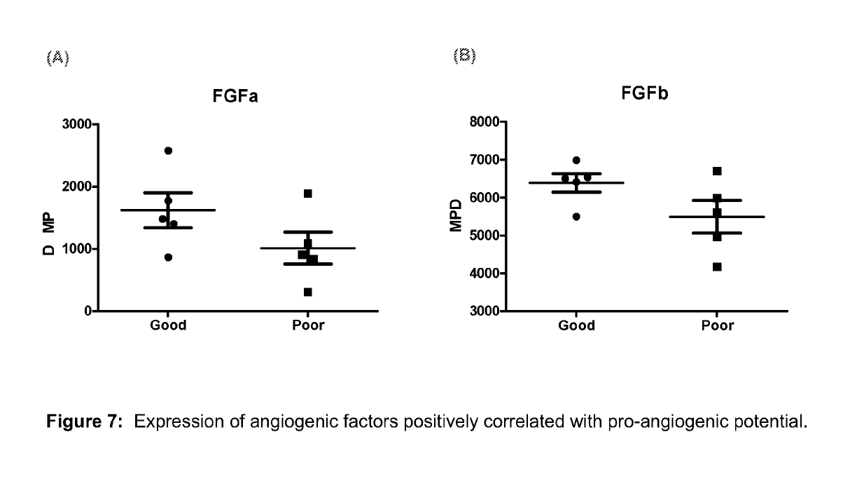

FIG. 7: Differential expression of angiogenic proteins that correlate positively with pro-angiogenic potential of cardiac-derived cells: FGFa (FIG. 7A) and FGFb (FIG. 7B).

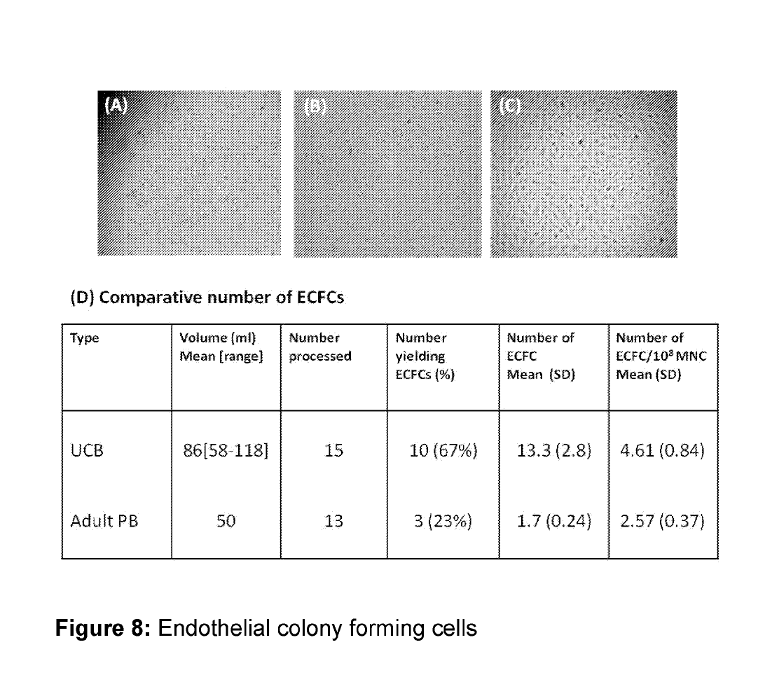

FIG. 8: Endothelial colony-forming cells (ECFC)/late outgrowth cells from different sources. ECFC/late outgrowth cells were obtained from umbilical cord blood (UCB) and adult peripheral blood (PB) by plating mononuclear cells on collagen coated plates. Endothelial-like colonies (FIG. 8A) were counted and isolated by plastic cloning rings and further expanded in culture to passage 2 (FIG. 8B). ECFC/late outgrowth cells grow as a cobblestone-like monolayer characteristic of endothelial cells such as HUVEC (FIG. 8C). A comparative analysis of ECFC obtained from UCB and adult PB shows that the number of ECFC/late outgrowth cells isolated from adult PB are lower that from UCB (FIG. 8D).

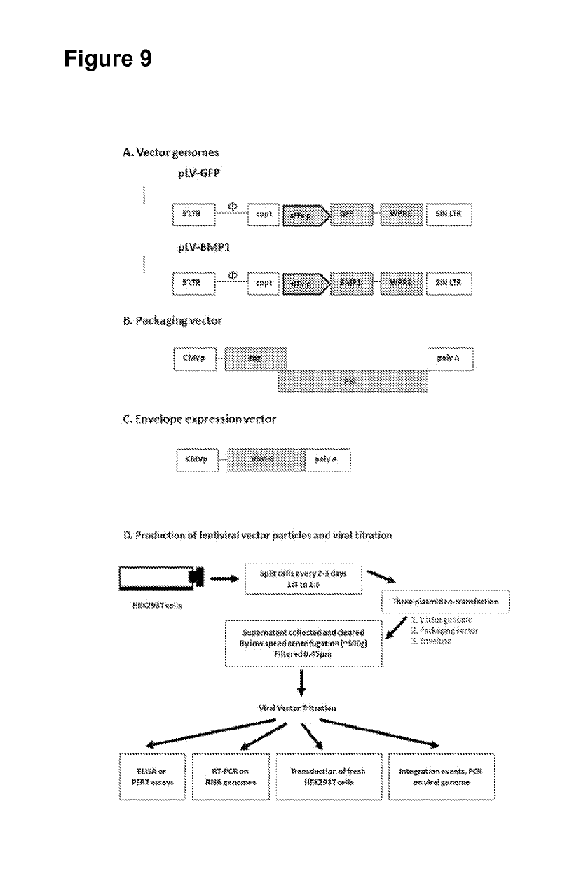

FIG. 9: Schematic diagram of the HIV-based lentiviral vector system used for GFP and BMP1 expression. Lentiviral vector genomes expressing the Green Fluorescent Protein (LV-GFP) or bone morphogenetic protein-1 (LV-BMP1) are shown (FIG. 9A) together with the lentiviral packaging vector (FIG. 9B) and the VSV-G envelope construct (FIG. 9C). LV-GFP and LV-BMP1 particles were produced using the three plasmid co-transfection method and titrated by standard methods on target cells (FIG. 9D).

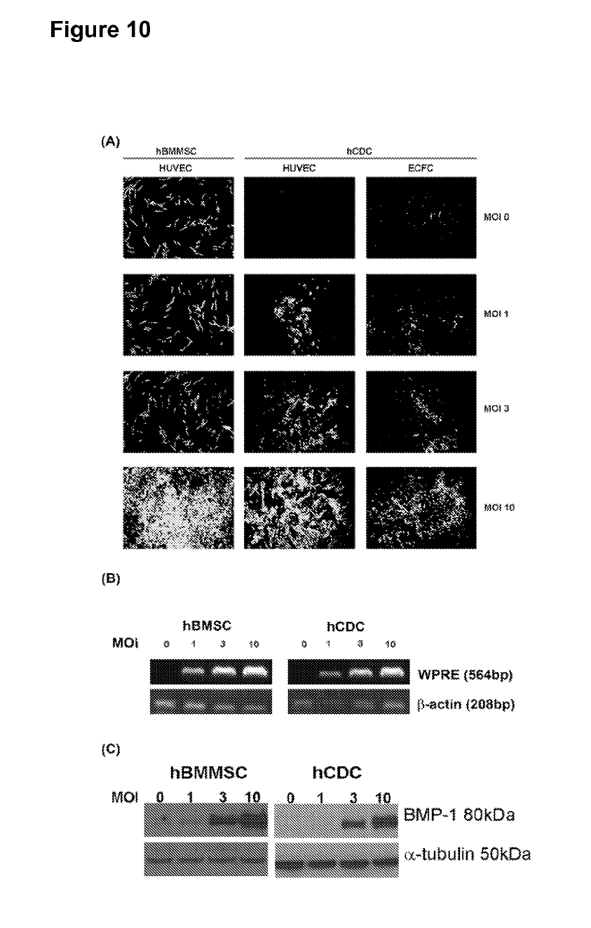

FIG. 10: Method for expanding endothelial cells ex vivo. Endothelial cells such as HUVEC or ECFC/late outgrowth cells obtained from UCB were labelled with a lentiviral vector expressing GFP. GFP-labelled cells were co-cultured with supportive cells, either cardiac-derived stromal cells (CDCs; Fig. A) or bone marrow-derived mesenchymal stromal cells (BMSC; Fig. A) transduced with LV-BMP1 at increasing multiplicity of infection (M.O.I. 1 to 10). The number of ‘green’ HUVEC and ECFC/late outgrowth cells increased significantly in cultures where the supportive cells expressed BMP1 compared to the mock transduced cells (M.O.I.=0). A confirmation that LV-BMP1 genomes were inserted in the cell host chromosomes was carried out by amplifying a 564 bp DNA fragment corresponding to the WPRE element present in the LV genome from total genomic DNA of the transduced cells. The presence of WPRE in the transduced cells and its absence in the mock transduced cells (M.O.I.=0) confirms integration of LV-BMP1 genomes (FIG. 10B). Amplification of a 208 bp DNA fragment of the β-actin gene was used as control in the integration assays. As expected, transduced cells expressed increasing amounts of BMP1 protein. Total cell extracts of human BMSC or CDCs transduced with LV-BMP1 were probed with BMP1 specific antibodies and detected by Western blotting using HRP-labelled secondary antibodies (FIG. 10C). α-tubulin was used as loading control in the Western blot.

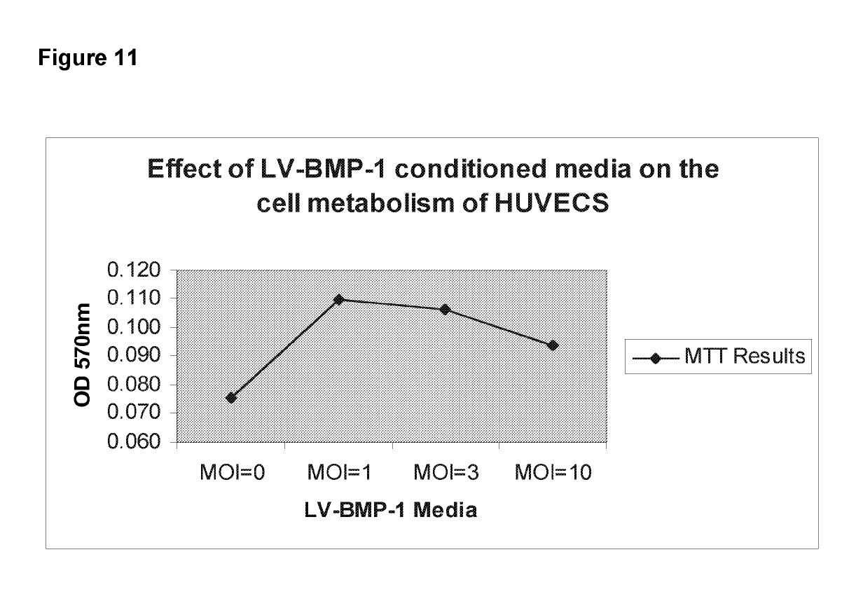

FIG. 11: Increased endothelial cell metabolism. HUVEC were incubated with conditioned media from cells transduced with LV-BMP1 at increasing M.O.I. (1 to 10) for 2-4 days and compared with untransduced controls. Cells were incubated with MTT for 3-4-h followed by DMSO. Absorbance at 570 nm was measured in treated and untreated HUVEC. An increase in absorbance at 570 nm showed an increase in metabolic rate of the cells when they are cultured in the presence of BMP-1.

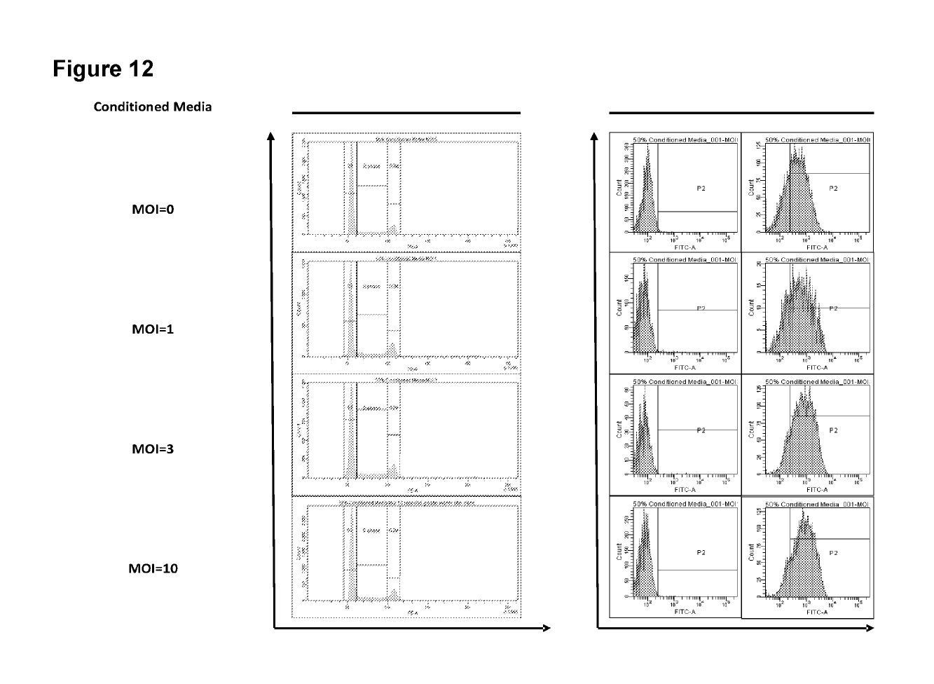

FIG. 12: Enhancement of endothelial cell proliferation. HUVEC were incubated with conditioned media from cells transduced with LV-BMP1 at increasing M.O.I. (1 to 10) for 2-4 days. Cells were then harvested and counted. For cell cycle analysis, cells were permeabilised and stained with propidium iodide (P1) and DAPI. The DNA content in the cells was analysed by flow cytometry. The proportion (%) of cells in G1, S or G2/M phases of the cell cycle was determined. For cell proliferation assays, treated and untreated cells were fixed in 70% Ethanol and stained either with an isotype control antibody or Ki67 antibody. The incorporation of Ki67 to HUVEC was analysed by flow cytometry.

### EXAMPLES

### Example 1

### Isolation, Expansion and Characterisation of Human Cardiac Stem/Progenitor Cells

Human cardiac progenitor cells were isolated and expanded ex vivo by their capacity to form cardiospheres without prior cell isolation. Cells were isolated from tissue biopsies (atrial and ventricular) from patients undergoing off-pump cardiac surgery according to established protocols ((Smith, Barile et al. 2007) and see FIG. 1A). Phase-bright cells were shed over a monolayer of stromal-like cells 7-14 days after plating. These cells form 3D structures called cardiospheres when cultured in the presence of cytokines. The number of phase bright cells and number and size of the cardiospheres was recorded as a function of the number of cells plated. The population doubling times and cell growth rates of cardiosphere derived cells (CDCs) was determined. Phenotypic characterisation by flow cytometry was performed (see FIG. 1D). CDCs from atrial (black) and ventricular (grey) biopsies lack the expression of haematopoietic (CD45) and endothelial (CD31) markers, but were positive for CD90 and CD105 (mesenchymal markers). A proportion of the cells also expressed CD117 (c-kit), a stem cell marker. Cardiac-derived cells capable of forming cardiospheres and with the ability to promote angiogenesis were found to be enriched in atrial biopsies compared to ventricular samples. Differentiated CDCs from two patients (osteo-, adipo- and chondrogenic lineages) appeared to be similar to control bone marrow mesenchymal progenitors (BMSC) (FIG. 1E). The expression of GATA 4 (FIG. 1F) and ACTA 2 (α-smooth muscle actin, FIG. 1G) was assessed by RT-PCR in five patients as examples of cardiac and smooth muscle cell markers. Expression was reported relative to β-2-microglobulin, a house-keeping gene (in percentage, %). Total mRNA from bone marrow mesenchymal stromal cells (BMSC), human umbilical vein endothelial cells (HUVEC), umbilical cord blood (UBC) CD133-positive cells and human heart were used as controls for RT-PCR analysis.

The results demonstrated that cardiosphere-forming cells are enriched in the atrium of the human heart and that these cells resemble mesenchymal progenitors in their expression of lineage-specific markers and differentiation potential.

### Example 2

### Assessing the Formation or Support of Vascular Networks

The vascular and mesenchymal potential of CDCs compared with EPC/MSC derived from blood or bone marrow was investigated using an established in vitro model of angiogenesis (Zhang et al., 2009). Human endothelial cells were transduced with VSV-G pseudotyped lentiviral vectors expressing GFP Cells at low multiplicity of infection (MOI 1 to 3) to ensure >95% transduction efficiency without detrimental effect on cell proliferation (Martin-Rendon, unpublished results). The transduced cells were co-cultured with supportive cells during 14 days in endothelial growth media that allows the formation of vascular tubular structures. The development of tubular structures was monitored during this period. The number of tubules formed, total tubules, average tubule length and number of junctions were determined using the AngioSys software package (TSC biologicals).

The results (FIG. 2) show that BMSC can support the formation of tubular structures resembling blood vessels in vitro (FIG. 2A). CDC can also support blood vessel formation (FIGS. 2B and 2C), however significant differences between patient samples have been observed. Representative images of blood vessels supported by CDC with ‘strong’ (patient 26—Pt 26) and ‘weak’ (patient 25—Pt 25) pro-angiogenic potential are shown in FIGS. 2D and 2E, respectively.

### Example 3

### Expression of Pro-Collagen Type I and Pro-Angiogenic Potential

Experiments to investigate the expression of pro-collagen type I in patients with strong and weak angiogenic potential demonstrated differences in expression (FIGS. 2F and 2G). Pro-collagen type I immunoreactivity in CDC obtained from 10 patients is significantly different between the ‘weak’ and ‘strong’ groups (FIG. 2G). These results demonstrate that human CDCs support the formation of vascular structures in vitro and that their pro-angiogenic potential inversely correlates with the expression of extracellular matrix components and deposition.

### Example 4

### Pro-Angiogenic Potential of Cardiac-Derived Cells (CDCs) from Patients with Ischaemic Heart Disease

Further experiments were conducted to investigate the pro-angiogenic ability of cardiac-derived cells isolated from ten different patients with ischaemic heart disease. Bone marrow-derived mesenchymal cells (BMSC) and CDCs obtained from 10 patients were co-cultured with GFP-labelled human umbilical vein endothelial cells (HUVEC) in endothelial growth media for 14 days. Blood vessel formation was monitored over 14 days and images were taken using a Nikon TE 300 microscope and PCI software. The number of tubules, junctions and total tubule length were quantitated using the Angiosys software (TCS Cellworks, England). The patient cells were divided into two distinct groups: strong and weak according to their pro-angiogenic ability. CDCs derived from patients 2, 8, 10, 23 and 26 exhibited strong angiogenic potential, whereas CDCs derived from patients 13, 14, 25, 27 and 28 exhibited weak angiogenic potential (FIG. 3A).

### Example 5

### Expression of Bone Morphogenetic Protein I and Pro-Angiogenic Potential

The expression of bone morphogenetic protein (BMP) 1 protein was investigated in the CDCs obtained from the 10 patients with ischaemic heart disease and in BMSC. Cells were grown under the same conditions as above and cell lysates were prepared. Approximately 30 pg total protein was loaded onto an SDS-PAGE gel. Proteins were transferred to nylon membranes and the membranes probed with anti-human BMP-1 antibody (R&D systems) and an anti-tubulin antibody (FIG. 3B). Relative levels of BMP1 protein were measured by densitometry, as shown in FIG. 3C. BMP1 protein levels in the patient samples were normalised to BMP1 expression in BMSC (from healthy donors-white bar). In nine out of ten patients there was a positive correlation between pro-angiogenic potential and BMP1 protein levels. Generally, CDCs with strong pro-angiogenic potential (black bars) have BMP-1 levels equal to or above the median level (˜75% marked by the horizontal line). In contrast, CDCs with weak pro-angiogenic potential (grey bars) showed BMP1 protein levels below the median. The exception is Pt 28. Taken together these results indicate that there is a correlation between the pro-angiogenic potential of CDCs obtained from patients with chronic ischaemic heart disease and BMP1 protein levels in those cells.

The influence of BMP1 on angiogenesis was investigated further using inhibitors. As above, BMSC and CDCs obtained from two patients (Pt 05 and Pt 07) were co-cultured with GFP-labelled HUVEC in endothelial growth media for 14 days. Cultures were treated with 10 uM BMP1 inhibitor during 14 days. Media was changed every three days and fresh inhibitor added. The number of tubules, junctions and total tubule length was quantitated as above using the Angiosys software. FIG. 4A shows the results for a healthy donor and two patients, where there is a significant reduction of tubules, junctions and tubule length in cells treated with the inhibitor ((*) p<0.05). FIG. 4B shows representative images of the co-cultures on day 14 in the absence and presence of BMP1 inhibitor. The inhibition of BMP1 activity has a negative effect on angiogenesis. Cells treated with BMP1 inhibitor have a reduced capacity to form blood vessels indicating that BMP1 is one of the factors that controls blood vessel formation.

The influence of BMP1 on angiogenesis was investigated further using small inhibiting RNAs. BMSC from a healthy donor and CDCs obtained from two patients (Pt 05 and Pt 07) were transfected with siRNA oligos using lipofectamine and according to the manufacturers' instructions. Approximately 24 hours post-transfection, cells were co-cultured with GFP-labelled HUVEC in endothelial growth medium for 4 and 7 days. FIG. 5A shows relative angiogenesis in cells treated with either non-target control (control-white bars) or specific BMP1 (dark bars) siRNA oligos. There is a significant reduction ((*)p, 0.05) in the number of tubules, junctions and total tubule length in the cultures treated with BMP1 siRNA compared to those treated with non-target control siRNA. FIG. 5B shows representative images of vascular network taken at day 4. As the expression of siRNA was transient, the cultures were monitored only for 4 and 7 days. It is noticeable that the vascular network is therefore not developed as well as the networks depicted in FIG. 4B (taken at day 14). Nevertheless, the formation of tubular structures was evident. The specific reduction of BMP1 expression in pro-angiogenic cells decreased angiogenesis suggesting that BMP1 may be one of the factors that modulates angiogenesis in the heart.

### Example 6

### Expression of Further Angiogenic Factors and Pro-Angiogenic Potential

The data suggest that there might be at least two groups of patients, those with ‘high’ and those with ‘low’ angiogenesis potential. Taking the data together there seem to be an inverse correlation between the ability of cardiac-derived cells to support angiogenesis and the expression of some extracellular matrix components (e.g. pro-collagen type I) in those cells (FIG. 2). There is also a correlation between cell proliferation and pro-angiogenic ability (Martin-Rendon, unpublished results).

Cardiac-derived cells from different patients with ‘good’ or ‘poor’ pro-angiogenic potential were grown in endothelial growth medium, under standard conditions to support the development of tubular structures. Cells were then spun down and cell lysates prepared as described previously. Total protein was quantitated by standard techniques. Cell lysates were used to probe a protein array (Proteome profiler array, from R&D Systems) where selected capture antibodies against 55 angiogenesis related proteins were spotted in duplicate on nitrocellulose membranes. The membranes were blocked for non-specific antibody binding prior to incubating them with approximately 100 μg of total protein. Any analyte complex bound to the immobilised capture antibody on the membrane was then detected using Streptavidin-Horseradish Peroxidase (Strep-HRP) and chemiluminescent detection reagents. The chemiluminescent signal produced was directly correlated to the amount of analyte bound. The positive signal was developed on an X-ray film and further scanned to quantitate using image analysis software. The mean pixel density (MPD) was determined by averaging the signal of the pair of duplicate spots representing each angiogenesis-related protein and subtracting the signal form negative control spots. The results obtained are shown in FIGS. 6 and 7, where expression of ADAMTS-1 (FIG. 6A), Angiogenin (FIG. 6B), Angiopoitin-1 (FIG. 6C), Angiostatin (FIG. 6D), EGF (FIG. 6E), EG-VEGF (FIG. 6F), Endostatin (FIG. 6G), PDGF-AA (FIG. 6H), PDGF-AB (FIG. 6I), Prolactin (FIG. 6J), TIMP-1 (FIG. 6K), and uPA (FIG. 6L) negatively correlate with the cells pro-angiogenic potential (i.e. where the expression of these factors was higher in cells exhibiting weak angiogenic potential). Expression of FGFa (FIG. 7A) and FGFb (FIG. 7B) positively correlate with pro-angiogenic potential of cardiac-derived cells (i.e. where the expression of these factors was higher in cells exhibiting strong angiogenic potential).

### Example 7

### Isolation of Endothelial Colony Forming Cells (ECFCs) from Blood

ECFCs/late outgrowth cells were isolated from umbilical cord blood (UCB) or adult peripheral blood (PB). Generally, 50-120 ml of UCB or 50 ml of PB were used. The mononucleated cell (MNC) fraction present in UCB and PB was isolated by ficoll density gradient centrifugation, according to previously described protocols (Zhang et al., 2009). MNCs were plated at a density of 2×10⁶ MNC per well in collagen coated 6×well plates and cultured in endothelial growth medium, such as EGM-2, for 2-4 weeks. Endothelial-like colonies (FIG. 8A) were identified and counted. Individual colonies were isolated with plastic cloning rings and expanded further in endothelial growth medium to confluency (FIG. 8B). The UCB and PB ECFC form a cobblestone-like monolayer characteristic of endothelial cells such as HUVEC (Figure C). The number of ECFCs obtained from UCB and adult PB are shown in FIG. 8D.

### Example 8

### Method for Expanding Endothelial Cells and ECFCS

Endothelial cells (EC) and endothelial colony forming cells (ECFC) were isolated ex vivo according to Example 7 from umbilical cord (HUVEC) or from the MNC fraction from umbilical cord blood or adult peripheral blood (ECFC) MNCs were plated in collagen coated plates and co-cultured with supportive cells. The EC or ECFC may be contacted with one or more angiogenic-related factor, preferably BMP-1, during culture in endothelial growth conditions and maintained for at least one week or more, preferably two weeks. The angiogenesis-related factor may be added to the cell culture media as a recombinant polypeptide or protein or produced by a supportive cell. The EC or ECFC may be contacted with conditioned media in which supportive cells have been previously cultured. The supportive cells may have been genetically modified to produce such factors during a short or a long term using a viral vector system or may be treated with an agent that modifies the expression of such angiogenesis-related factors.

The data suggest that the number of ECs and ECFCs in adult human PB is low. One of the challenges to use them in large quantities in therapeutic angiogenesis is to expand these cells ex vivo. The data presented suggest that there is a direct positive correlation between angiogenesis and levels of BMP-1 protein expression in the supportive cells (FIG. 3). The cDNA from the human BMP-1 gene was obtained from LabOmics (GeneCopoeia, Rockville, Md., USA) in the mammalian expression vector pEZ-M09. BMP-1 cDNA was digested with appropriate restriction enzymes to excise it from the parental plasmid and cloned into a plasmid containing a lentiviral vector (LV) genome (kindly provided by Prof. Adrian Thrasher) with minor modifications. In this system, human BMP-1 cDNA is expressed under the control of the spleen focus forming virus (sFFv) promoter in the vector genome (FIG. 9A). LV-BMP1 viral particles were produced using the three plasmid co-transfection methods (FIG. 9D) (Soneoka et al., 1995) using the same packaging lentiviral vector plasmid (FIG. 9B) and the same VSV-G expression plasmid (FIG. 9C) as the one used for GFP above. Particles were obtained from the supernatant of HEK293T cells approximately 48 h post-transfection (FIG. 9D). LV-BMP1 viral stocks were tittered in parallel with LV-GFP viral stocks. Approximately 2×10⁶ to 2×10⁷ transducing units per mL were obtained. Supportive stromal cells either derived from bone marrow (BMSC) or from cardiac tissue (CDCs) were transduced at different M.O.I. ranging from 1 to 10. Two days after transduction supportive cells were co-cultured with GFP-labelled HUVEC or UCB ECFCs for 2 weeks. The number of green ECFCs and HUVEC (FIG. 10A) increased significantly in cultures where the supportive cells express BMP1 compared with mock transduced cells (FIG. 10A).

### Example 9

### Enhancement of Endothelial Cell Metabolism

Bone marrow- or cardiac-derived mesenchymal/stromal cells were transduced with LV-BMP1 particles at increasing M.O.I. (MOI 1 to 10) as described in Example 8. Approximately 48 h post-transduction the media was changed and left for another 48 h. The conditioned media of transduced and non-transduced cells was collected, cleared of cell debris by low speed centrifugation and used to culture HUVEC. Endothelial cell metabolic state was determined using standard assays such as MTT assay. HUVEC were seeded in 96×well plates at 500 cells per well. 200 μl of conditioned media was added to the cells either on its own or mixed with fresh media in a 1:1 ratio. HUVEC were incubated with the conditioned media for 2-4 days after which cells were treated with 50 μl MTT/well and incubated at 37° C. for 3-4-h. The supernatant was aspirated and approximately 100 μl DMSO added to the wells. Absorbance at 570 nm gives a reflection of the metabolic state of the cells compared with control.

Treatment of HUVEC with conditioned media of cells transduced with BMP-1 significantly increases absorbance at 570 nm, indicating that BMP-1 containing conditioned media increases HUVEC metabolism (FIG. 11).

### Example 10

### Enhancement of Endothelial Cell Proliferation

Bone marrow- or cardiac-derived mesenchymal/stromal cells were transduced with LV-BMP1 particles at increasing M.O.I. (MOI 1 to 10) and the conditioned media of transduced and non-transduced cells was used to culture HUVEC and determine its effect on HUVEC proliferation. HUVEC were seeded in 6×well plates at approximately 5×10⁴ cells per well and incubate with 2 ml of conditioned media for 2-4 days at 37° C. and 5% CO₂. Cells were then harvested and counted. For cell cycle analysis, cells were permeabilised with 0.1% Triton X-100 and stained with propidium iodide (PI) and DAPI according to previously published protocols (Pozarowski and Darzyhiewicz). The DNA content of HUVEC treated cells was analysed by flow cytometry (FIG. 12). The representative histograms show the percentage of cells in G1, S or G2/M phases of the cells cycle for each condition. The results suggest that HUVEC treated with conditioned media containing BMP-1 showed an increase in the proportion of cells in G2/M. This proportion increased from 13.6% in cells treated with conditioned media from transductions at MOI=0 to 18.1% in cells treated with media from transductions at MOI=1. This increase in G2/M is maintained when cells are treated with media from transductions at MOI=3 and 10.

In order to determine cell proliferation, a second method was also used. Cells were fixed in 70% Ethanol for 2 h. Cells were washed in phosphate buffered saline and resuspended at 10⁷ cells/ml in 100 μl. Cells were then incubated for 30 mins with either an isotype control antibody or Ki67 antibody, both conjugated to FITC. FITC-conjugated Ki67 bound to the cells was detected by flow cytometry as indicated in FIG. 12. The results suggest that there is an increase in the percentage of Ki67-positive cells when HUVEC are treated with conditioned media from transduced cells compared to non-transduced cells. Taken together these results suggest that endothelial cell metabolism, cell cycle progression and cell proliferation or/and cell survival are promoted when these cells are cultured in the presence of BMP-1. The angiogenesis-related factors may increase the metabolism of EC or ECFC, induce the proliferation of EC or ECFC, or enhance their cell survival.

### REFERENCES

1) Freedman, S. B., and Isner, J. M; Therapeutic Angiogenesis for Coronary Artery Disease; Annals of Internal Medicine 2002; 136(1): 54-71.
2) Rivard A, Fabre J E, Silver M, Chen D, Murohara T, Kearney M, et al. Age-dependent impairment of angiogenesis. Circulation. 1999; 99:111-20.
3) Rivard A, Silver M, Chen D, Kearney M, Magner M, Annex B, et al. Rescue of diabetes-related impairment of angiogenesis by intramuscular gene therapy with adeno-VEGF. Am J. Pathol. 1999; 154:355-63.
4) Couffinhal T, Silver M, Kearney M, Sullivan A, Witzenbichler B, Magner M, et al. Impaired collateral vessel development associated with reduced expression of vascular endothelial growth factor in ApoE−/− mice. Circulation. 1999; 99: 3188-98.
5) Schultz A, Lavie L, Hochberg I, Beyar R, Stone T, Skorecki K, et al. Interindividual heterogeneity in the hypoxic regulation of VEGF: significance for the development of the coronary artery collateral circulation. Circulation. 1999; 100:547-552.
6) Martin-Rendon, E., Snowden, J. A., and Watt, S. M. Stem cell-related therapies for vascular diseases. Transfusion Medicine. 2009; 19(4):159-171.
7) Passaniti A, Taylor R M, Pili R, Guo Y, Long P V, Haney J A, Pauly R R, Grant D S, Martin G R. A simple, quantitative method for assessing angiogenesis and antiangiogenic agents using reconstituted basement membrane, heparin, and fibroblast growth factor, Lab Invest. 1992 October; 67(4):519-28.
8) Antibodies: A Laboratory Manual (Harlow and Lane Eds. Cold Spring Harbor Press)
9) Smith, R. R., Barile, L., Cho, N. C., Leppo, M. K., Hare, J. M., Messina, E., Giacomello, A., Abraham, M. R., and Marban, E. Regenerative Potential of Cardiosphere-Derived Cells Expanded From Percutaneous Endomyocardial Biopsy Specimens. Circulation. 2007; 115:896-908.
10) Zhang, Y., Fisher, N., Newey, S. E., Smythe, J., Tatton, L., Tsaknakis, G., Forde, S. P., Carpenter, L., Athanassopoulos, T., Hale, S. J., Ferguson, D. J., Tyler, M. P., and Watt, S. M. The impact of proliferative potential of umbilical cord-derived endothelial progenitor cells and hypoxia on vascular tubule formation in vitro. Stem Cells Dev. 2009 March; 18(2):359-75.
11) Watt, S. M., Athanassopoulos, A., Harris, A. L. and Tsaknakis, G. Human endothelial stem/progenitor cells, angiogenic factors and vascular repair. J R Soc Interface. 2011, 7 (Suppl 6): S731-51.
12) Critser P. J. and Yoder M. C. Endothelial colony-forming cell role in neoangiogenesis and tissue repair. Curr. Opin. Organ Transpl. 2010, 15: 68-72.
13) Estes M. L., Mund J. A., Ingram D. A. and Case J. Identification of endothelial cells and progenitor cell subsets in human peripheral blood. Curr. Protoc, Cytom. 2010, 52: 9.33.1-9.33.11.
14) Fischer C., Schneider M. and Carmeliet P. Principles and therapeutic implications of angiogenesis, vasculogenesis and arteriogenesis. Handb. Exp. Pharmacol. 2006, 176: 157-212.
15) Soneoka, Y.; Cannon, P. M.; Ramsdale, E. E.; Griffiths, J. C.; Romano, G.; Kingsman, S. M. and Kingsman, A. J'A transient three-plasmid expression system for the production of high titer retroviral vectors' Nucleic Acids Res 1995, 23(4): 628-33

1.-43. (canceled)

44. A method for determining the angiogenic signature of a cell or tissue sample comprising obtaining an ex vivo cell or tissue sample from a subject, (a) testing the sample for the expression levels of at least three angiogenesis-related factors selected from the group consisting of: ADAMTS-1, Angiogenin, Angiopoietin-1, Angiostatin, BMP-1, EGF, EGVEGF, Endostatin, FGFa, FGFb, PDGF-AA, PDGF [symbol] AB, Prolactin, TIMP-1, pro-collagen type I and uPA, (b) comparing the results determined in step (a) with reference levels of expression; and (c) predicting the pro-angiogenic potential of the subject from the outcome of the comparison in step (b).

45. The method as claimed in 44, wherein increased levels of one or more of ADAMTS-1, Angiogenin, Angiopoitin-1, Angiostatin, EGF, EG-VEGF, Endostatin, PDGF-AA, PDGFAB, Prolactin, TIMP-1, pro-collagen type I and uPA compared to reference levels negatively correlate with pro-angiogenic potential.

46. The method as claimed in 44, wherein increased levels of one or more of FGFa, FGFb, BMP-1 compared to reference levels positively correlate with pro-angiogenic potential.

47. The method for predicting efficacy of angiogenic therapy, comprising: determining the proangiogenic potential of a subject using the method as claimed in claim 44 and matching the pro-angiogenic potential with a model profile correlated to angiogenic treatment regimen and subject outcome.

48. A method for preparing a predictive model for predicting angiogenic potential, comprising: with ex vivo cell or tissue samples obtained from a plurality of subjects, (a) testing each sample for the expression levels of at least three angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, procollagen type I and uPA; (b) testing the each sample in an in vitro and/or in vivo angiogenesis assay; and (c) matching the results from step (a) with the results from step (b).

49. A method for identifying an agent for use in angiotherapy, comprising: with an ex vivo cell or tissue sample obtained from a subject, (a) contacting the sample with the agent, (b) testing the sample for the presence and/or levels of at least three angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA; (c) comparing the presence and/or levels of expression of the angiogenesis-related factors with the presence and/or levels of expression of the same angiogenesis-related factors in a sample that has not been contacted with the agent.

50. The method as claimed in claim 44 wherein the sample is obtained from the subject's heart or vascular tissue, Wharton's Jelly, umbilical cord, umbilical cord blood, peripheral blood, bone marrow, embryonic cells, endothelial cells or iPS cells or from an atrial or ventricular biopsy, or a biopsy from a venous or arterial blood vessel.

51. The method as claimed in claim 50 wherein the sample comprises skeletal, cardiac, endothelial or stromal supportive cells or mesenchymal or haematopoietic stem or progenitor cells, embryonic cells or iPS cells or iPS-derived endothelial cells or cardiac-derived progenitor cells or stem cells, cardiomyocytes or associated cardiac fibroblasts or vascular cells or a combination of these cells.

52. The method as claimed in claim 44 wherein the testing comprises measuring gene expression at the RNA and/or protein level.

53. The method as claimed in claim 44 wherein prior to testing, cells from the sample are expanded in cell culture.

54. The method as claimed in claim 44, wherein at least five of ADAMTS [symbol] 1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA are tested.

55. The method as claimed in claim 44, wherein all of ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA are tested.

56. A method for improving the pro-angiogenic potential of cells comprising: with an ex vivo cell or tissue sample obtained from a subject (a) testing the sample for the expression levels of at least three angiogenesis-related factors selected from the group consisting of: ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA; (b) comparing the results determined in step (a) with reference levels of expression; (c) predicting the pro-angiogenic potential of the subject from the outcome of the comparison in step (b), optionally expanding cells from the sample in cell culture and (d) treating the cells using one or more of (i) hypoxia preconditioning, (ii) pharmacological treatment and (iii) genetic modification.

57. The method as claimed in claim 56, further comprising transplanting the treated cells into the subject.

58. A method for expanding endothelial cells (EC) and endothelial colony forming cells (ECFC) ex vivo comprising the steps of contacting EC and ECFC with one or more angiogenesis-related factors during culture in endothelial growth conditions.

59. The method according to claim 58 wherein the angiogenesis-related factor is a recombinant polypeptide or protein.

60. The method according to claim 58 wherein supportive cell are provided.

61. The method according to claim 60 wherein the supportive cells are selected from stromal cells, bone marrow cells, cardiac tissue cells, umbilical cord blood cells, Wharton's Jelly, perivascular cells, adipose tissue, microvascular-derived mesenchymal/stromal cells, adult and embryonic skin fibroblasts, embryonic stem (ES) cells or induced pluripotent stem (iPS) derived supportive stromal cells.

62. The method according to claim 58 wherein the ECs and ECFCs are obtained from an embryo, a neonate, an adult, or tissues such as Wharton's Jelly-, umbilical cord-, umbilical cord blood-, peripheral blood-, large or small blood vessels-, microvasculature-, ES or iPS derived endothelial cell, UCB, PB or vasculature.

63. The method according to claim 58 wherein the angiogenesis-related factor is selected from ADAMTS-1, Angiogenin, Angiopoietin-1, Angiostatin, BMP-1, EGF, EGVEGF, Endostatin, FGFa, FGFb, PDGF-AA, PDGF-AB, Prolactin, TIMP-1, pro-collagen type I and uPA.

64. The method according to claim 58 wherein the ECs and the ECFC are modified to produce angiogenesis-related factors selected from ADAMTS-1, Angiogenin, Angiopoietin-1, Angiostatin, BMP-1, EGF, EG-VEGF, Endostatin, FGFa, FGFb, PDGF-AA, PDGF-AB, Prolactin, TIMP-1, pro-collagen type I and uPA.

65. Use of expanded EC or ECFCs produced by the methods described in claim 58, BMP-1, FGFa, FGFb or a combination thereof for the preparation of a medicament for the treatment of an ischaemic condition.

66. Use according to claim 65 wherein the ischemic condition is selected from coronary artery disease (CAD), peripheral arterial disease (PAD), stroke, cerebral ischaemia, wound healing, liver, lung or renal ischaemia.

67. A medical implant comprising one or more of expanded EC, ECFC prepared by the method defined in claim 58, BMP-1, FGFa and FGFb.

68. A pharmaceutical composition comprising a pharmaceutically acceptable diluent, carrier or excipient and one or a combination of expanded EC, ECFC according to the method described in claim 58, BMP-1, FGFa and FGFb.
